(19) United States
(12) Patent Application Publication
(10) Pub. No.: US 2019/0284609 A1
(43) Pub. Date: Sep. 19, 2019

(54) FAST, MULTIPLEX AMPLIFICATION OF NUCLEIC ACIDS

(71) Applicant: LUMINEX CORPORATION, Austin, TX (US)

(72) Inventors: Douglas WHITMAN, Round Rock, TX (US); Johanna TAKACH, Austin, TX (US)

(73) Assignee: LUMINEX CORPORATION, Austin, TX (US)

(21) Appl. No.: 16/353,385

(22) Filed: Mar. 14, 2019

Related U.S. Application Data
(60) Provisional application No. 62/643,088, filed on Mar. 14, 2018.

(51) Int. Cl. C12Q 1/686; C12Q 1/6813

(52) U.S. Cl. CPC C12Q 1/686; C12Q 1/6813; C12Q 2600/16; C12Q 2537/143; C12Q 2531/113

(57) ABSTRACT

Method of nucleic acid amplifying are provided. In some aspects, methods involve amplifying at least two different target nucleic acids in a reaction mixture.

This application claims the benefit of U.S. Provisional Patent Application No. 62/643,088, filed Mar. 14, 2018, the entirety of which is incorporated herein by reference.

### BACKGROUND OF THE INVENTION

### 1. Field of the Invention

The present invention relates generally to the field of molecular biology. More particularly, it concerns methods for identifying and quantifying nucleic acid targets in biological samples.

### 2. Description of Related Art

Detection of target nucleic acids from biological or environmental samples often utilizes amplification strategies to amplify either the amount of target nucleic acid in the sample or the signal resulting from a detection scheme in order to achieve detection of limited amounts of target nucleic acid in the sample. One commonly used nucleic acid amplification strategy is the polymerase chain reaction (PCR). In typical PCR reactions, multiple cycles of denaturation of duplex nucleic acids, annealing of primers to target nucleic acids and extension of hybridized primers are performed in order to yield detectable levels of target nucleic acids. While denaturation, annealing and extension occur rapidly once critical temperatures for these biological interactions are reached, the ability to rapidly cycle a reaction between temperatures ranging from 95° C., typically used for denaturation, and 50-60° C., typically used for annealing, and 72° C. typically used for extension is limited by the instrumentation available to achieve rapid changes in temperature. Various instrumentation improvements have been made in efforts to speed this process. In addition, strategies have been developed to reduce the range of temperatures used in thermal cycling. For example, US 20170198342 describes using primers selected to have annealing temperatures that overlap with the denaturation temperature of the target amplicons to reduce the temperature difference required for primer annealing and amplicon denaturation steps, thus reducing the time required for cycling. Primer modifications to increase primer annealing temperatures include primers having 5′ non-target-specific portions and 3′ target-specific portions that generate amplicons that include sequences of the 5′ non-target specific tails. The addition of the 5′ non-target-specific portions to the amplicons permits a higher annealing temperature to be used than would be possible for a primer having only the 3′ target-specific portion. This serves to reduce the temperature difference between denaturation and annealing steps of the cycles. Further reduction of the temperature difference between denaturation and annealing steps can be achieved by reducing the denaturation temperature to reflect the required denaturation temperature of the amplified target rather than the denaturation temperature of the native target nucleic acid.

In many scenarios, it is desirable to include multiple sets of primers capable of amplifying different target nucleic acids in a single reaction. For example, in diagnostic applications in which infectious disease agents are sought to be identified, multiple primer sets, each specific for a different infectious disease agent are included in a single reaction designed for use with a single patient sample. Primer sets for such multiplex applications should be designed to have similar annealing temperatures in order to effectively amplify all target nucleic acids in the sample under a similar set of amplification conditions. However, designing suitable primer sets that uniformly amplify different targets present at low levels in a sample in under 30 minutes can be challenging.

It would be desirable to find improved methods for performing multiplex amplification that could also reduce the time required to achieve detectable amounts of amplified target nucleic acids in a reaction.

### SUMMARY OF THE INVENTION

In one embodiment, a method is provided for amplifying at least two different target nucleic acids in a reaction mixture, the method comprising: (a) adding to the reaction mixture a primer set specific to each different target nucleic acid, at least one primer of each set comprising a 5′ portion and a 3′ portion, the 5′ portion being non-complementary to any nucleic acid sequence in the reaction mixture and the 3′ portion being capable of specific hybridization to its respective nucleic acid target, wherein at least 2 primer sets have initial annealing temperatures for specifically hybridizing to their respective target nucleic acids that are at least 2 degrees different; (b) performing at least two cycles of amplification comprising: (i) heating the sample to a first denaturation temperature Td1 that denatures all different target nucleic acids in the sample; (ii) hybridizing the primers of each primer set to their respective denatured different target nucleic acids at a temperature Ta1 that is the same as or lower than the lowest initial annealing temperature of all sets of primers; (iii) extending the hybridized primers to form extension products; (c) performing at least 10 additional cycles of amplification by: (i) heating the sample to a second denaturation temperature Td2 that is lower than Td1 and denatures all extension products in the reaction mixture; (b) hybridizing the primers of each primer set to their respective denatured extension products at a temperature Ta2 that is higher than Ta1; (c) extending the hybridized primers.

In certain embodiments, after the at least two cycles of amplification the at least two primer sets hybridize to their respective extension products at annealing temperatures that are less than 2 degrees Celsius different, less than 1.5 degrees Celsius different, less than 1 degree Celsius different, or less than 0.5 degrees Celsius different. In some embodiments, Ta2 is the same as or lower than the lowest annealing temperature of any primer hybridized to its respective complementary extension product. In some embodiments Ta1 and Td1 differ by 18 degrees Celsius or more and Td2 and Ta2 differ by less than 18 degrees Celsius.

The certain embodiments the 5′ portions of the primers are selected such that primers having initial annealing temperatures that are at least 2 degrees Celsius different have annealing temperatures that are within a 2 degrees Celsius range, 1.5 degree Celsius range, 1.0 degree Celsius range, or 0.5 degree Celsius range of each other after the at least two cycles of amplification.

In another embodiment, a method is provided for amplifying at least two different target nucleic acids in a reaction mixture, the method comprising: (a) adding to the reaction mixture a primer set specific to each different target nucleic acid, at least one primer of each set comprising a 5′ portion and a 3′ portion, the 5′ portion being non-complementary to any nucleic acid sequence in the reaction mixture and the 3′ portion being capable of specific hybridization to its respective nucleic acid target, wherein at least 2 primer sets have initial annealing temperatures for specifically hybridizing to their respective target nucleic acids; (b) performing at least two cycles of amplification comprising: (i) heating the sample to a first denaturation temperature Td1 that denatures all different target nucleic acids in the sample; (ii) hybridizing the primers of each primer set to their respective denatured different target nucleic acids at a temperature Ta1 that is the same as or lower than the lowest initial annealing temperature of all sets of primers; (iii) extending the hybridized primers to form extension products; (c) performing at least 10 additional cycles of amplification by: (i) heating the sample to a second denaturation temperature Td2 that is lower than Td1 and denatures all extension products in the reaction mixture; (ii) hybridizing the primers of each primer set to their respective denatured extension products at a temperature Ta2 that is higher than Ta1; (iii) extending the hybridized primers.

In certain embodiments, before the at least two cycles of amplification the at least two primer sets hybridize to their respective target nucleic acids at annealing temperatures that are more than 2 degrees Celsius different and after the at least two cycles of amplification the at least two primer sets hybridize to their respective extension products at annealing temperatures that are less than 2 degrees Celsius different, less than 1.5 degrees Celsius different, less than 1 degree Celsius different, or less than 0.5 degrees Celsius different. In some embodiments, Ta1 and Td1 differ by 18 degrees Celsius or more and Td2 and Ta2 differ by less than 18 degrees Celsius.

In some embodiments, the 5′ portions of the primers are selected such that primers having initial annealing temperatures that are more than 2 degrees Celsius different have annealing temperatures that are within at least a 2 degrees Celsius range, 1.5 degree Celsius range, 1.0 degree Celsius range, or 0.5 degree Celsius range of each other after the at least two cycles of amplification. In some embodiments, Ta2 is the same as or lower than the lowest annealing temperature of any primer hybridized to its respective complementary extension product.

A primer is a nucleic acid that is capable of priming the synthesis of a nascent nucleic acid in a template-dependent process. A target-specific primer refers to a primer that has been designed to prime the synthesis of a particular target nucleic acid. As used herein, “hybridization,” “hybridizes” or “capable of hybridizing” is understood to mean the forming of a double or triple stranded molecule or a molecule with partial double or triple stranded nature. The term “anneal” as used herein is synonymous with “hybridize.” As used herein “stringent conditions” or “high stringency” are those conditions that allow hybridization between or within one or more nucleic acid strands containing complementary sequences, but preclude hybridization of non-complementary sequences. Such conditions are well known to those of ordinary skill in the art, and are preferred for applications requiring high selectivity. Stringent conditions may comprise low salt and/or high temperature conditions. It is understood that the temperature and ionic strength of a desired stringency are determined in part by the length of the particular nucleic acids, the length and nucleobase content of the target sequences, the charge composition of the nucleic acids, and to the presence or concentration of formamide, tetramethylammonium chloride or other solvents in a hybridization mixture.

As used herein, “essentially free,” in terms of a specified component, is used to mean that none of the specified component has been purposefully formulated into a composition and/or is present only as a contaminant or in trace amounts. The total amount of the specified component resulting from any unintended contamination of a composition is preferably below 0.01%. Most preferred is a composition in which no amount of the specified component can be detected with standard analytical methods.

As used herein in the specification and claims, “a” or “an” may mean one or more. As used herein in the specification and claims, when used in conjunction with the word “comprising”, the words “a” or “an” may mean one or more than one. As used herein, in the specification and claim, “another” or “a further” may mean at least a second or more.

As used herein in the specification and claims, the term “about” is used to indicate that a value includes the inherent variation of error for the device, the method being employed to determine the value, or the variation that exists among the study subjects.

Other objects, features and advantages of the present invention will become apparent from the following detailed description. It should be understood, however, that the detailed description and the specific examples, while indicating certain embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1: is a flow diagram illustrating one embodiment of a method for performing a rapid amplification reaction.

FIG. 2: is a graph representing the measured temperatures inside a PCR tube during thermal cycling.

FIGS. 3A-3C: FIG. 3A is a graph illustrating a Flu A melt profile. FIG. 3B is a graph illustrating a Flu B melt profile. FIG. 3C is a graph illustrating a RSV melt profile.

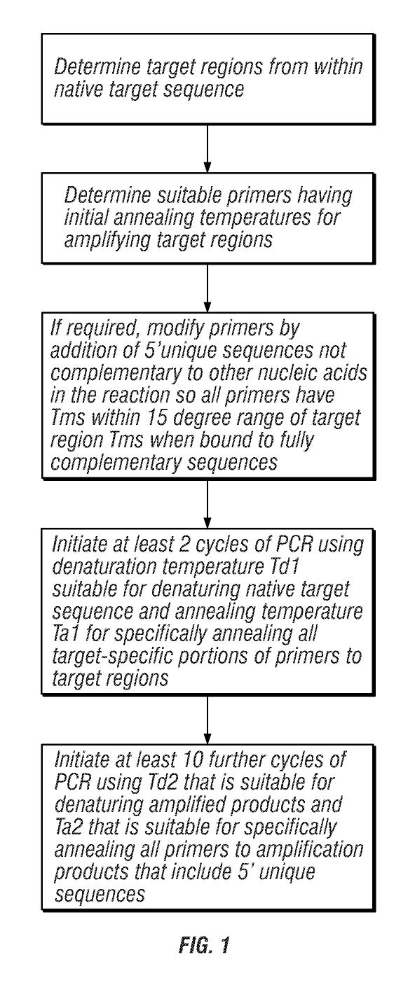

### DESCRIPTION OF ILLUSTRATIVE EMBODIMENTS

The present disclosure provides methods for performing rapid amplification reactions for determining the presence or absence of multiple target nucleic acids in a sample. In particular, the reactions include multiple different primer sets, each specific for a different target nucleic acid. When used in the disclosed method, amplification reactions are completed in 30 minutes or less using standard thermal cyclers, and yield highly specific amplification products.

In multiplex amplification reactions, it is desirable to use primers having similar annealing temperatures so that during PCR amplification, the annealing temperature can be set to achieve optimal specific binding of all primers to their respective target nucleic acid sequences. However, this can constrain primer design, as it can be challenging to design multiple different primer sets, each specific for a desired target sequence, and each having an annealing temperature within a 2 degree range.

When using primers having different annealing temperatures in a multiplex reaction, the annealing step must typically be performed at a temperature equivalent to or lower than the lowest primer annealing temperature of the primers in the mixture. However, use of lower than necessary annealing temperatures for the other primers in the mixture often results in non-specific annealing, yielding non-specific amplification products. Therefore, it is desirable to allow primers in the reaction to anneal at higher temperatures to optimize the generation of specific amplification products.

According to one embodiment, primers for different target nucleic acids are permitted to anneal to their targets at the temperature suitable for the primer having the lowest annealing temperature for at least two cycles of PCR. Subsequently, the annealing temperature of further amplification cycles is increased to ensure specific amplification products are synthesized. Primers having 5′ non-target complementary portions and 3′ target-specific portions are utilized. The 3′ target specific portions may anneal to their targets at different annealing temperatures. However, after at least 2 cycles of amplification, the 5′ portions are incorporated into the amplicons, and the primers are thus able to anneal to their respective amplification products at higher annealing temperatures determined by the annealing of both the 5′ and 3′ portions of the primer to the amplicon. The degree of change in annealing temperature of a primer to its target is determined by the composition and length of the sequence of the 5′ portion. Since the sequences of the 5′ portions are unrelated to the target sequences, the sequences can be carefully selected to complement their respective 3′ portions to ensure that all primers bind to extended amplicons at temperatures that are within a 2 degree range of each other. Either one or both primers of the primer set for amplifying each target can include a 5′ non-target specific portion. In this way, primers having initial annealing temperatures to native target nucleic acids (determined by hybridization of just their 3′ portions to their respective targets) that differ by more than 2 degrees, can, after 2 cycles of amplification, anneal to amplified products at a common, higher temperature than is used in early cycles of amplification. In this way, all target nucleic acids sequences in the reaction can be amplified simultaneously using a uniform set of cycling conditions. Furthermore, after the initial few rounds of amplification, the ratio of target amplicons to native target sequence changes as the number of extended primers in the reaction mixture increases. Early in the amplification reaction, when a limited number of amplicons are present, it is important to ensure that most, if not all, native target nucleic acids in the reaction are denatured to optimize the number of primers binding to their target sequences. Typically, this is achieved using denaturation temperatures of 95° C. As the ratio of amplicon: native target starts to increase, it becomes less important to denature native target sequences since primers are able to utilize amplicons as annealing templates. At this point, denatured amplicons can provide sufficient templates for primer binding. Since amplicons generally have lower denaturation temperatures than native target sequences, it is thus possible at this stage to reduce the denaturation temperature of the reaction to the minimum required for amplicon denaturation.

Detection of amplified products can be achieved by methods known in the art, either in real time as amplification proceeds or using end-point detection. For example, U.S. Pat. No. 7,541,147 describes a real-time detection method using a primer having a labeled non-natural base. Incorporation of a complementary labeled non-natural base during complementary strand synthesis results in a change in signal from the labels. Other real time detection schemes known in the art, such as that described in U.S. Pat. Nos. 5,804,375, 7,381,818 and US20160040219 may also be used. The use of probe-based detection schemes that utilize melt analysis to identify and distinguish different amplification products or probe-target interactions permits increased levels of multiplexing over those that rely only on differentially labeled amplification products or probes to distinguish different amplification products or probe-target interactions.

In contrast to the method for multiplexing taught in US20170198342, which teaches selecting primer sets that will amplify different targets under different cycling conditions to ensure that only 1 target nucleic acid is amplified per condition, the method of present invention provides for amplifying all target nucleic acids under uniform amplification conditions. An advantage of this method is that rather than requiring different cycling conditions for each different target in a sample, only two different sets of conditions are required to amplify all targets in a sample. Furthermore, the first set of conditions requires only 2 cycles, while the second set of conditions utilizes increased annealing and decreased denaturation temperatures as compared to the first two cycles to reduce cycle time. Thus, the present disclosure provides methods for amplifying multiple target nucleic acids in a fast, convenient format that requires fewer amplification cycles than that taught in US 20170198342.

### Example 1

### Flu A, Flu B, RSV Multiplex PCR

A test of Flu A, Flu B, and RSV primers along with a control primer set containing 5′ extensions was performed. Table 1 indicates the % GC content and melting temperature (Tm) of the primers and amplicon in the multiplex before adding the 5′ non-specific region and after adding the 5′ non-specific region. The original primers had greater variation in Tm compared to after modification. The 5′ modified primers had a lower delta temperature between the predicted Tm of the primers and the predicted Tm of the amplicon.

TABLE 1

| | | | | | | | 5′ | | Amplicon containing | | |
|---|---|---|---|---|---|---|---|---|---|---|---|
| | Original | | Original Amplicon | | | | modified | | 5′ modified primer | | |
| | Primers | | | | Amplicon | | primers | | | | Amplicon |
| Target | % GC | Tm | GC | Tm | Length | 5' modification | % GC | Tm | % GC | Tm | Length |
| Flu A | 47.1 | 55.2 | 53.8 | 84 | 80 | /56-FAM//iMe-isodC/CCTACCTCTCCTACCTCTC | 54.1 | 72.2 | 54 | 85 | 113 |
| | | | | | | T | | | | | |
| | 60 | 59.1 | | | | CTCACTCCTCCATCTC | 56.7 | 72.3 | | | |
| Flu B | 26.9 | 61.6 | 40.2 | 79 | 92 | /5TexRd-XN//iMe-isodC/ACTCACCTCTCCTCTC | 37.8 | 72.5 | 44.5 | 81 | 128 |
| | | | | | | TCA | | | | | |
| | 45 | 62.6 | | | | CCACTCCTCTCTCTCC | 54.1 | 72.4 | | | |
| RSV | 25 | 63.2 | 33.3 | 75 | 84 | /56-JOEN//iMe-isodC/CCACCACCTCTCCTCCT | 40 | 72.8 | 40.3 | 79 | 119 |
| A/B | 33.3 | 62.6 | | | | CTCCTCCTCTTCTACTCT | 40.5 | 72.4 | | | |
| MHV | 29.2 | 59.3 | 37 | 78.2 | 81 | /56-TAMN//iMe-isodC/CTTCCTCCTCCACTCT | 40 | 71.2 | 42.1 | 79.6 | 114 |
| | 40 | 58.3 | | | | CCATCCTCCTCATCTCT | 45.9 | 71.5 | | | |

Flu A, Flu B, RSV multiplex PCR mastermix was set up according to Table 2. MHV was used as an internal control. A multiplex mastermix was prepared using the 5′ modified primers, also following the mastermix calculation in Table 2. The mastermix was prepared for a 15 uL reaction volume. The Water (AM9937), 2M KCL (AM9640G), 0.5M MgCl2 (AM9530G) were obtained from Ambion Inc. The TiTaq (S1792) was obtained from Clonetec Inc. The Ultramer DNA samples for Flu A Flu B and RSV were diluted 1:2 in resuspension buffer.

TABLE 2

| | | RXN |
|---|---|---|
| | | 20 |
| Reagent | per 15 uL rxn (uL) | Total Vol. (uL) |
| Water | 0.691 | 13.83 |
| 10X ISOlution | 1.875 | 37.50 |
| 2M KCl | 0.469 | 9.38 |
| .5M MgCl2 | 0.098 | 1.95 |
| Conjugated Duplex Inhibitor | 0.300 | 6.00 |
| RSV | 1.500 | 30.00 |
| Flu A | 1.500 | 30.00 |
| Flu B | 1.500 | 30.00 |
| MHC | 1.125 | 22.50 |
| MHC Target | 0.375 | 7.50 |
| MMLV | 0.068 | 1.35 |
| TiTaq Polymerase | 1.500 | 30.00 |
| Target DNA | 4.000 | — |
| ABI rxn vol. | 15.000 | |
| rxn vol. check | 15.000 | |

The test was performed on an instrument that can perform PCR heating and cooling on 4 PCR tubes and read raw fluorescent data in 6 fluorescent wavelengths. The PCR profile and the final melt temperature profile was performed on the same instrument. The PCR profile and melt were performed on tubes 1 and 2 of the instrument. A small stainless steel ball was placed in the PCR tubes before addition of mastermix. 11 uL of mastermix was added to the PCR tubes 1 and 2. 15 uL Water was added to PCR tubes 3 and 4. 4 uL of the sample was added to PCR tube 1 and 4 uL of water was added to tube 2 for the No Template Control Sample (NTC). All primers in the reaction were denatured and extended simultaneously in each phase of the thermal cycling reaction.

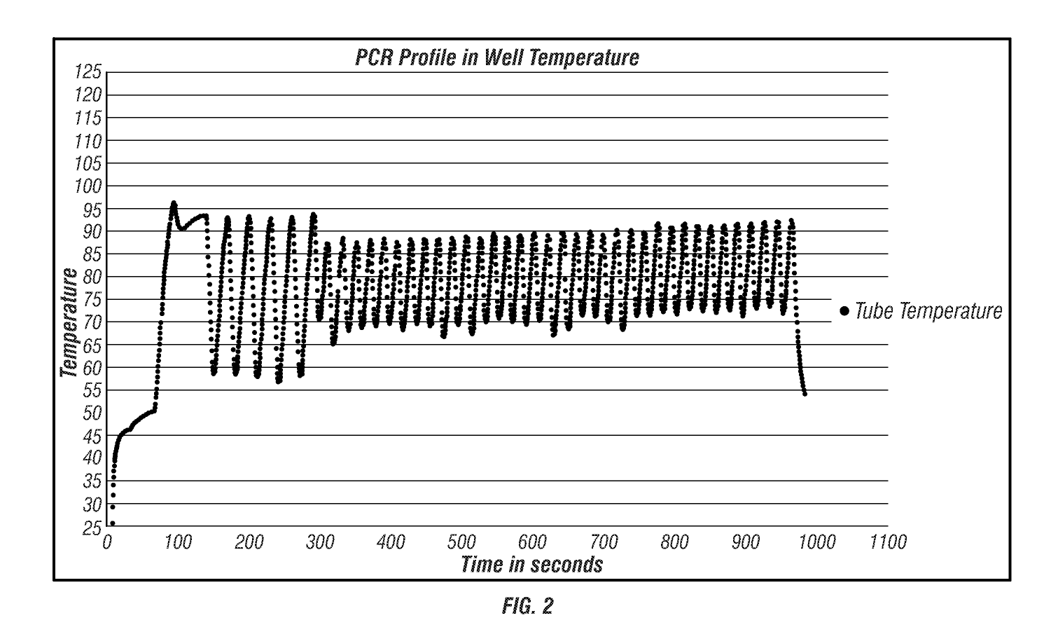

The instrument PCR profile shown in Table 3 was performed with 5 cycles of a high delta temperature between anneal and denature, followed by many cycles of a lower delta temperature between anneal and denature steps. The difference between the anneal and denature temperatures was 18° C. FIG. 2 is a graph representing the measured temperatures inside the PCR tube as the cycling proceeded.

TABLE 3

| | | | Set |
|---|---|---|---|
| PCR Step | # of Cycles | Hold Time | Temperature |
| Reverse Transcription Step | 1 | 120 sec | 50° C. |
| Hot start Taq Activation | 1 | 90 sec | 95° C. |
| High Delta Cycling Anneal | 5 | 1 sec | 58° C. |
| High Delta Cycling Denature | | 1 sec | 93° C. |
| Low Delta Cycling Anneal | 35 | 1 sec | 70° C. |
| Low Delta Cycling Denature | | 1 sec | 88° C. |

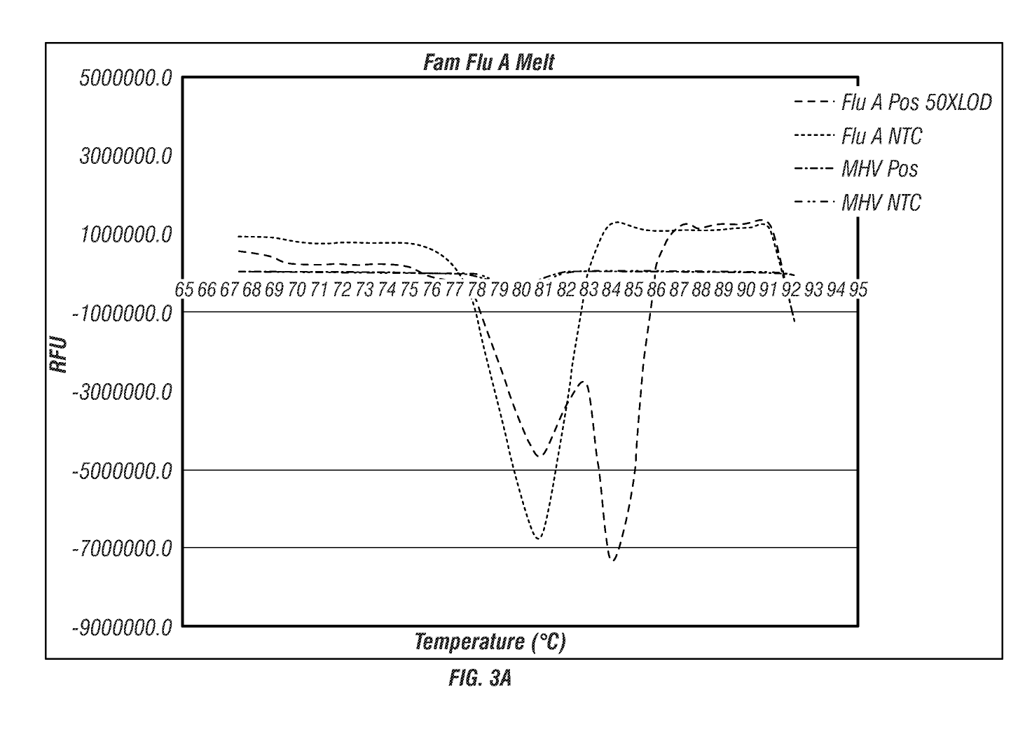

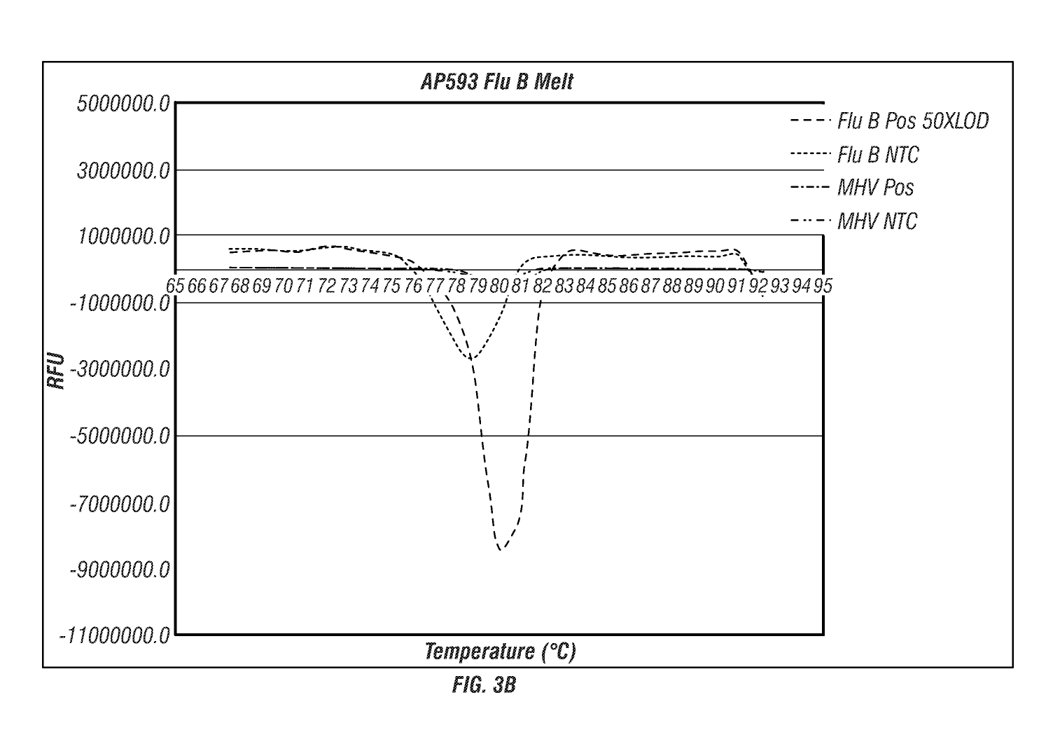

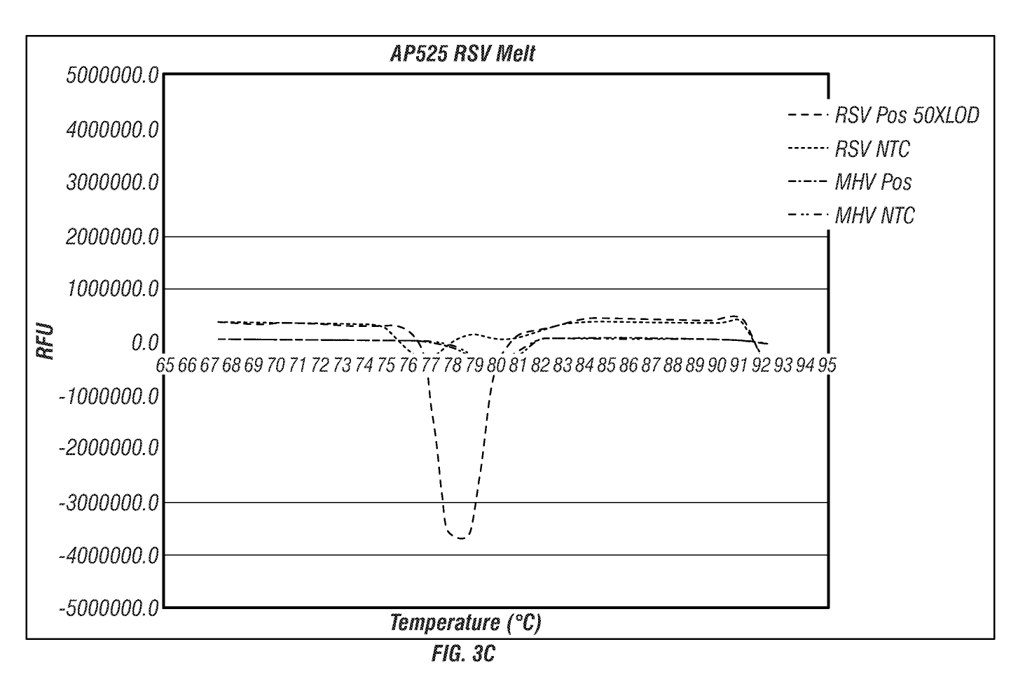

The graphs in FIGS. 3A, 3B, and 3C demonstrate a melt analysis performed after amplification that confirmed positivity of the target in the multiplex reaction by the expected Tms of the amplicons that were generated during the amplification process.

1. A method of amplifying at least two different target nucleic acids in a reaction mixture, the method comprising:
a) Adding to the reaction mixture a primer set specific to each different target nucleic acid, at least one primer of each set comprising a 5′ portion and a 3′ portion, the 5′ portion being non-complementary to any nucleic acid sequence in the reaction mixture and the 3′ portion being capable of specific hybridization to its respective nucleic acid target, wherein at least 2 primer sets have initial annealing temperatures for specifically hybridizing to their respective target nucleic acids that are at least 2 degrees different,
b) Performing at least two cycles of amplification comprising:
i. Heating the sample to a first denaturation temperature Td1 that denatures all different target nucleic acids in the sample;
ii. Hybridizing the primers of each primer set to their respective denatured different target nucleic acids at a temperature Ta1 that is the same as or lower than the lowest initial annealing temperature of all sets of primers;
iii. extending the hybridized primers to form extension products;
c) Performing at least 10 additional cycles of amplification by:
i. Heating the sample to a second denaturation temperature Td2 that is lower than Td1 and denatures all extension products in the reaction mixture;
ii. Hybridizing the primers of each primer set to their respective denatured extension products at a temperature Ta2 that is higher than Ta1;
iii. Extending the hybridized primers.

2. The method of claim 1 wherein after the at least two cycles of amplification the at least two primer sets hybridize to their respective extension products at annealing temperatures that are less than 2 degrees Celsius different.

3. The method of claim 1 wherein Ta2 is the same as or lower than the lowest annealing temperature of any primer hybridized to its respective complementary extension product.

4. The method of claim 1 wherein Ta1 and Td1 differ by more than 18 degrees Celsius and Td2 and Ta2 differ by less than 18 degrees Celsius.

5. The method of claim 1 wherein the 5′ portions of the primers are selected such that primers having initial annealing temperatures that are at least 2 degrees Celsius different have annealing temperatures that are within a 2 degree Celsius range of each other after the at least two cycles of amplification.

6. A method of amplifying at least two different target nucleic acids in a reaction mixture, the method comprising:
a) Adding to the reaction mixture a primer set specific to each different target nucleic acid, at least one primer of each set comprising a 5′ portion and a 3′ portion, the 5′ portion being non-complementary to any nucleic acid sequence in the reaction mixture and the 3′ portion being capable of specific hybridization to its respective nucleic acid target, wherein at least 2 primer sets have initial annealing temperatures for specifically hybridizing to their respective target nucleic acids,
b) Performing at least two cycles of amplification comprising:
i. Heating the sample to a first denaturation temperature Td1 that denatures all different target nucleic acids in the sample;
ii. Hybridizing the primers of each primer set to their respective denatured different target nucleic acids at a temperature Ta1 that is the same as or lower than the lowest initial annealing temperature of all sets of primers;
iii. extending the hybridized primers to form extension products;
c) Performing at least 10 additional cycles of amplification by:
i. Heating the sample to a second denaturation temperature Td2 that is lower than Td1 and denatures all extension products in the reaction mixture;
ii. Hybridizing the primers of each primer set to their respective denatured extension products at a temperature Ta2 that is higher than Ta1;
iii. Extending the hybridized primers.

7. The method of claim 6 wherein before the at least two cycles of amplification the at least two primer sets hybridize to their respective target nucleic acids at annealing temperatures that are more than 2 degrees Celsius different and after the at least two cycles of amplification the at least two primer sets hybridize to their respective extension products at annealing temperatures that are less than 2 degrees Celsius different.

8. The method of claim 6 wherein Ta1 and Td1 differ by more than 18 degrees Celsius and Td2 and Ta2 differ by less than 18 degrees Celsius.

9. The method of claim 6 wherein the 5′ portions of the primers are selected such that primers having initial annealing temperatures that are more than 2 degrees Celsius different have annealing temperatures that are within at least a 2 degree Celsius range of each other after the at least two cycles of amplification.

10. The method of claim 6 wherein Ta2 is the same as or lower than the lowest annealing temperature of any primer hybridized to its respective complementary extension product.
